(19) United States
(12) Patent Application Publication
(10) Pub. No.: US 2014/0363856 A1
(43) Pub. Date: Dec. 11, 2014

(54) AN IMPROVED PRE-HYDROLYSIS STEP INVOLVING VACUUM

(71) Applicant: Beta Renewables S.p.A., Tortona, AL (IT)

(72) Inventors: Edwin Andrew Sisson, Medina, OH (US); Simone Ferrero, Tortona (AL); Paolo Torre, Arenzano (IT); Piero Ottonello, Milano (IT); Francesco Cherchi, Novi Ligure (IT); Giuseppe Grassano, San Giuliano Vecchio (IT); Luis Oriani, Itatiba (BR); Dario Giordano, Tortona (IT)

(73) Assignee: Beta Renewables S.p.A., Tortona, AL (IL)

(21) Appl. No.: 14/362,986

(22) PCT Filed: Dec. 20, 2012
(86) PCT No.: PCT/EP2012/076419

§ 371 Date: Jun. 5, 2014

(30) Foreign Application Priority Data
Jan. 11, 2012 (IT) TO2012A000012

(51) Int. Cl. C12P 19/02

(52) U.S. Cl. CPC C12P 19/02
USPC 435/105

(57) ABSTRACT

An improved pre-hydrolysis step involving exposing water insoluble pre-treated ligno-cellulosic biomass to vacuum conditions, with and without enzymes is disclosed. After exposing the water insoluble pre-treated ligno-cellulosic biomass to vacuum conditions, enzymatic hydrolysis is conducted on the pre-treated material. The result is an increased yield of glucose and often xylose after the enzymatic hydrolysis when compared to a composition which has not been exposed to vacuum conditions.

### BACKGROUND

Both US 2009/0053777 A1 and WO 2009/046538 A1 both consider the use of vacuum in various parts of a biomass conversion process.

In order of the processing steps, US 2009/0053777 A1 discloses a Pretreatment and Enzymatic Hydrolysis Reactor to which vacuum and pressure may be applied to the reaction vessel by attaching external sources to the lance-connected port in the cover.

US 2009/0053777 A1 further discloses a large barrel piston reactor of 5.1 cm×68.6 cm stainless steel barrel equipped with a piston, oriented horizontally. The 68.6 cm barrel was equipped with eight multiple use ports allowing application of vacuum, injection of aqueous ammonia, injection of steam and insertion of thermocouples for measurement of temperature inside the barrel. The reactor barrel was directly attached to a 15.2 cm×61 cm stainless steel flash tank, oriented vertically. The pre-treated solids were directed down into the bottom of the flash tank where the solids were easily removed by unbolting a domed end flange in the bottom of the tank.

The use of the vacuum is disclosed when a vacuum was applied to the reactor vessel and to the flash receiver to bring the pressure down <10 kPa, and dilute ammonium hydroxide solution was injected in the reactor. Once the ammonia was charged, steam was injected into the reactor to bring the temperature to 145° C. The mixture was then discharged into the preheated flash tank by activating the piston. Vacuum was pulled on the flash tank until the flash receiver reached ˜59° C. Upon harvest from the flash receiver, free liquid was separated from the pre-treated solids and not added back for saccharification.

WO 2009/046538 A1, titled ENZYMATIC TREATMENT UNDER VACUUM OF LIGNOCELLULOSIC MATERIALS, is self descriptive. The enzymatic hydrolysis of the ligno-cellulosic biomass is done under vacuum so as to remove the inhibitors to further the enzymatic reaction.

The use of vacuum in these references is for very specific reasons and under very specific conditions. Neither of these references disclose or render non-inventive, the process and the efficiencies described in the description portion of this specification.

### SUMMARY

Disclosed in this specification is an improved pre-hydrolysis step involving vacuum with one embodiment comprising the steps of

A) Exposing a composition to a vacuum condition,
wherein the composition has a dry matter content, and
the composition comprises a water insoluble pre-treated ligno-cellulosic biomass produced from a ligno-cellulosic biomass processed in a pre-treatment process, and an added liquid which has been added to the water insoluble pre-treated ligno-cellulosic biomass after the pre-treatment process,
wherein the weight percent of the dry matter content of the composition by weight of the total amount of the composition is in the range of 1 to 60 weight percent;

B) Ceasing to expose the composition to the vacuum condition,

C) Adding at least one catalyst to the composition wherein the catalyst is capable of hydrolyzing the water insoluble pre-treated ligno-cellulosic biomass in the composition,

D) Conducting a catalytic hydrolysis of the water insoluble pre-treated ligno-cellulosic biomass in the composition.

In another embodiment, the composition is void of free liquid. In another embodiment, the composition comprises free liquid.

It is further disclosed that the step of exposing the composition to a vacuum condition and the step of conducting a catalystic hydrolysis are not conducted in the same vessel.

It is further disclosed that the vacuum condition can be less than an absolute pressure measured in millibar (mbar) selected from the group consisting of 950, 900, 850, 800, 700, 600, 500, 400, 300, 250, 200, 150, 100, 50, 30, 20, 10, 5, and 0.5 mBar.

It is further disclosed that the weight percent of dry matter of the composition by weight of the total amount of the composition can be in a range selected from the group consisting of 1 to 50, 1 to 40, 1 to 36, 1 to 30, 1 to 25, 1 to 20, 1 to 15, 1 to 10, and 5 to 40.

It is also disclosed that the step of exposing the composition to the vacuum condition may include maintaining the exposure of the composition to the vacuum condition for a minimum time selected from the group consisting of 5 minutes, 10 minutes, 20 minutes, 30 minutes, 45 minutes, and 60 minutes.

It is also disclosed that the exposure to the vacuum condition may be conducted in a temperature range consisting of a temperature range selected from the group consisting of 15 to 55° C., 15 to 50° C., 15 to 45° C., 15 to 35° C., and 15 to 30° C.

It is further disclosed that the composition and/or the added liquid may be void of a catalyst capable of hydrolyzing the water insoluble pre-treated ligno-cellulosic biomass. It is also disclosed that the catalyst may comprise an enzyme and that the catalytic hydrolysis may be enzymatic hydrolysis.

It is further disclosed that the added liquid may comprise C5's which were separated from the water insoluble pre-treated ligno-cellulosic biomass as part of the pre-treatment of the water insoluble pre-treated ligno-cellulosic biomass.

It is also disclosed that the added liquid may also comprise a hydrolysis product made from the enzymatic hydrolysis of a similarly composed water insoluble pre-treated ligno-cellulosic biomass.

It is further disclosed that the step of exposing the composition to the vacuum condition may be conducted using a cylinder with a screw inside the cylinder, also known as an extruder.

It is also disclosed that the conducting of the catalytic hydrolysis is not done under any vacuum condition.

That the process may be continuous is also disclosed and that the composition be void of ammonia and the pre-treatment process may be void of ammonia.

### BRIEF DESCRIPTION OF THE FIGURES

FIG. 1 compares the amount of xylose and glucose generated over time by the enzymatic hydrolysis of a composition comprising a water insoluble pre-treated ligno-cellulosic biomass which has been exposed to a vacuum condition prior to enzymatic hydrolysis with a water insoluble pre-treated ligno-cellulosic biomass of the same composition which has not been exposed to a vacuum condition prior to enzymatic hydrolysis at the specified enzyme concentration.

FIG. 2 compares the amount of xylose and glucose generated over time by the enzymatic hydrolysis of a composition comprising a water insoluble pre-treated ligno-cellulosic biomass which has been exposed to a vacuum condition prior to enzymatic hydrolysis with a water insoluble pre-treated ligno-cellulosic biomass of the same composition which has not been exposed to a vacuum condition prior to enzymatic hydrolysis at the specified enzyme concentration.

FIG. 3 compares the amount of xylose and glucose generated over time by the enzymatic hydrolysis of a composition comprising a water insoluble pre-treated ligno-cellulosic biomass which has been exposed to a vacuum condition prior to enzymatic hydrolysis with a water insoluble pre-treated ligno-cellulosic biomass of the same composition which has not been exposed to a vacuum condition prior to enzymatic hydrolysis at the specified enzyme concentration.

FIG. 4 compares the amount of xylose and glucose generated over time by the enzymatic hydrolysis of a composition comprising a water insoluble pre-treated ligno-cellulosic biomass which has been exposed to a vacuum condition prior to enzymatic hydrolysis without enzymes with a water insoluble pre-treated ligno-cellulosic biomass of the same composition has been exposed to a vacuum condition with enzymes prior to enzymatic hydrolysis at the specified enzyme concentration.

FIG. 5 compares the relative amount of xylose and glucose generated over time by the enzymatic hydrolysis of a composition comprising a water insoluble pre-treated ligno-cellulosic biomass which has been exposed to a vacuum condition prior to enzymatic hydrolysis without enzymes with a water insoluble pre-treated ligno-cellulosic biomass of the same composition has not been exposed to a vacuum condition prior to enzymatic hydrolysis at the specified enzyme concentration.

### DESCRIPTION

This specification discloses a process to increase the recovery of glucose from a water insoluble pre-treated ligno-cellulosic biomass by applying vacuum to a composition comprising the water insoluble pre-treated ligno-cellulosic biomass for a short period of time. As disclosed below, the composition comprising the water insoluble ligno-cellulosic biomass may further include an added liquid (also referred to as an added first liquid), free liquid, or be void of free liquid.

What has been discovered and discussed in the experimental section is that when a water insoluble pre-treated ligno-cellulosic biomass is exposed to a vacuum condition under a liquid, such as water, the water insoluble pre-treated ligno-cellulosic biomass swells and expands to about 140% of its original volume and then, once the entrained gas of the water insoluble pre-treated ligno-cellulosic biomass is released, it collapses back to about 80% of its original volume. While vacuum under liquid is a preferred embodiment, exposing the composition comprising the water insoluble ligno-cellulosic biomass, but void of free liquid or added liquid to a vacuum condition is another embodiment of the invention.

The experiments establish that, contrary to the previous art, catalysts, such as enzymes for enzymatic hydrolysis, are not necessary during the vacuum step to further penetrate the water insoluble pre-treated ligno-cellulosic biomass. The enzymes or other hydrolysis catalysts such as acids or bases can be added after the vacuum is broken. The yield of the sugars is the same, whether the vacuum is conducted under water or under water with enzymes.

The experiments also establish that the vacuum step is preferably conducted under or in a liquid, preferably water. Experiments performed on the water insoluble pre-treated ligno-cellulosic biomass without adding liquid, or in the absence of a free liquid, had much lower sugar yields than those experiments where the vacuum was applied on the water insoluble pre-treated ligno-cellulosic biomass in the presence of an amount of liquid. While it is preferred to expose the composition to the vacuum condition in the presence of liquid, or under a liquid, the exposure of the composition without liquid is still better than not exposing the composition to vacuum at all.

The experimental data also establishes that the step of conducting catalytic hydrolysis such as enzymatic hydrolysis under vacuum can be avoided if the vacuum is applied prior to catalytic hydrolysis, such as enzymatic hydrolysis, even if only for 10 minutes.

With this knowledge experimentally established, the process therefore comprises first, exposing a composition to a vacuum condition. A suitable composition comprises a water insoluble pre-treated ligno-cellulosic biomass. To be a water insoluble pre-treated ligno-cellulosic biomass means that at least a portion of the biomass is water insoluble and that the original naturally occurring ligno-cellulosic biomass used to derive the water insoluble pre-treated ligno-cellulosic biomass has undergone processing (pre-treatment) to change its chemical or physical characteristics from that found in nature.

The first step of creating a water insoluble pre-treated ligno-cellulosic biomass is to use a ligno-cellulosic biomass. A preferred ligno-cellulosic biomass can be described as follows: Apart from starch, the three major constituents in plant biomass are cellulose, hemicellulose and lignin, which are commonly referred to by the generic term lignocellulose. Polysaccharide-containing biomasses as a generic term include both starch and lignocellulosic biomasses. Therefore, some types of feedstocks can be plant biomass, polysaccharide containing biomass, and ligno-cellulosic biomass.

Polysaccharide-containing biomasses according to the present invention include any material containing polymeric sugars e.g. in the form of starch as well as refined starch, cellulose and hemicellulose.

Relevant types of naturally occurring biomasses for deriving the claimed invention may include biomasses derived from agricultural crops selected from the group consisting of starch containing grains, refined starch; corn stover, bagasse, straw e.g. from rice, wheat, rye, oat, barley, rape, sorghum; softwood e.g. Pinussylvestris, Pinus radiate; hardwood e.g. Salix spp. Eucalyptus spp.; tubers e.g. beet, potato; cereals from e.g. rice, wheat, rye, oat, barley, rape, sorghum and corn; waste paper, fiber fractions from biogas processing, manure, residues from oil palm processing, municipal solid waste or the like. Although the experiments are limited to a few examples of the enumerated list above, the invention is believed applicable to all because the characterization is primarily to the unique characteristics of the lignin and surface area.

The ligno-cellulosic biomass feedstock used in the process is preferably from the family usually called grasses. The proper name is the family known as Poaceae or Gramineae in the Class Liliopsida (the monocots) of the flowering plants. Plants of this family are usually called grasses, or, to distinguish them from other graminoids, true grasses. Bamboo is also included. There are about 600 genera and some 9,000-10,000 or more species of grasses (Kew Index of World Grass Species).

Poaceae includes the staple food grains and cereal crops grown around the world, lawn and forage grasses, and bamboo. Poaceae generally have hollow stems called culms, which are plugged (solid) at intervals called nodes, the points along the culm at which leaves arise. Grass leaves are usually alternate, distichous (in one plane) or rarely spiral, and parallel-veined. Each leaf is differentiated into a lower sheath which hugs the stem for a distance and a blade with margins usually entire. The leaf blades of many grasses are hardened with silica phytoliths, which helps discourage grazing animals. In some grasses (such as sword grass) this makes the edges of the grass blades sharp enough to cut human skin. A membranous appendage or fringe of hairs, called the ligule, lies at the junction between sheath and blade, preventing water or insects from penetrating into the sheath.

Grass blades grow at the base of the blade and not from elongated stem tips. This low growth point evolved in response to grazing animals and allows grasses to be grazed or mown regularly without severe damage to the plant.

Flowers of Poaceae are characteristically arranged in spikelets, each spikelet having one or more florets (the spikelets are further grouped into panicles or spikes). A spikelet consists of two (or sometimes fewer) bracts at the base, called glomes, followed by one or more florets. A floret consists of the flower surrounded by two bracts called the lemma (the external one) and the palea (the internal). The flowers are usually hermaphroditic (maize, monoecious, is an exception) and pollination is almost always anemophilous. The perianth is reduced to two scales, called lodicules, that expand and contract to spread the lemma and palea; these are generally interpreted to be modified sepals.

The fruit of Poaceae is a caryopsis in which the seed coat is fused to the fruit wall and thus, not separable from it (as in a maize kernel).

There are three general classifications of growth habit present in grasses; bunch-type (also called caespitose), stoloniferous and rhizomatous.

The success of the grasses lies in part in their morphology and growth processes, and in part in their physiological diversity. Most of the grasses divide into two physiological groups, using the C3 and C4 photosynthetic pathways for carbon fixation. The C4 grasses have a photosynthetic pathway linked to specialized Kranz leaf anatomy that particularly adapts them to hot climates and an atmosphere low in carbon dioxide.

C3 grasses are referred to as “cool season grasses” while C4 plants are considered “warm season grasses”. Grasses may be either annual or perennial. Examples of annual cool season are wheat, rye, annual bluegrass (annual meadowgrass, Poaannua and oat). Examples of perennial cool season are orchard grass (cocksfoot, Dactylisglomerata), fescue (Festucaspp), Kentucky Bluegrass and perennial ryegrass (Loliumperenne). Examples of annual warm season are corn, sudangrass and pearl millet. Examples of Perennial Warm Season are big bluestem, indiangrass, bermuda grass and switch grass.

One classification of the grass family recognizes twelve subfamilies: These are 1) anomochlooideae, a small lineage of broad-leaved grasses that includes two genera (Anomochloa, Streptochaeta); 2) Pharoideae, a small lineage of grasses that includes three genera, including Pharus and Leptaspis; 3) Puelioideae a small lineage that includes the African genus Puelia; 4) Pooideae which includes wheat, barley, oats, brome-grass (Bronnus) and reed-grasses (Calamagrostis); 5) Bambusoideae which includes bamboo; 6) Ehrhartoideae, which includes rice, and wild rice; 7) Arundinoideae, which includes the giant reed and common reed 8) Centothecoideae, a small subfamily of 11 genera that is sometimes included in Panicoideae; 9) Chloridoideae including the lovegrasses (Eragrostis, ca. 350 species, including teff), dropseeds (Sporobolus, some 160 species), finger millet (Eleusinecoracana (L.) Gaertn.), and the muhly grasses (Muhlenbergia, ca. 175 species); 10) Panicoideae including panic grass, maize, sorghum, sugar cane, most millets, fonio and bluestem grasses; 11) Micrairoideae; 12) Danthoniodieae including pampas grass; with Poa which is a genus of about 500 species of grasses, native to the temperate regions of both hemispheres.

Agricultural grasses grown for their edible seeds are called cereals. Three common cereals are rice, wheat and maize (corn). Of all crops, 70% are grasses.

Sugarcane is the major source of sugar production. Grasses are used for construction. Scaffolding made from bamboo is able to withstand typhoon force winds that would break steel scaffolding. Larger bamboos and Arundo donax have stout culms that can be used in a manner similar to timber, and grass roots stabilize the sod of sod houses. Arundo is used to make reeds for woodwind instruments, and bamboo is used for innumerable implements.

The ligno-cellulosic biomass feedstock may also be from woody plants or woods. A woody plant is a plant that uses wood as its structural tissue. These are typically perennial plants whose stems and larger roots are reinforced with wood produced adjacent to the vascular tissues. The main stem, larger branches, and roots of these plants are usually covered by a layer of thickened bark. Woody plants are usually either trees, shrubs, or lianas. Wood is a structural cellular adaptation that allows woody plants to grow from above ground stems year after year, thus making some woody plants the largest and tallest plants.

These plants need a vascular system to move water and nutrients from the roots to the leaves (xylem) and to move sugars from the leaves to the rest of the plant (phloem). There are two kinds of xylem: primary that is formed during primary growth from procambium and secondary xylem that is formed during secondary growth from vascular cambium.

What is usually called “wood” is the secondary xylem of such plants.

The two main groups in which secondary xylem can be found are:
1) conifers (Coniferae): there are some six hundred species of conifers. All species have secondary xylem, which is relatively uniform in structure throughout this group. Many conifers become tall trees: the secondary xylem of such trees is marketed as softwood.
2) angiosperms (Angiospermae): there are some quarter of a million to four hundred thousand species of angiosperms. Within this group secondary xylem has not been found in the monocots (e.g Poaceae). Many non-monocot angiosperms become trees, and the secondary xylem of these is marketed as hardwood.

The term softwood is used to describe wood from trees that belong to gymnosperms. The gymnosperms are plants with naked seeds not enclosed in an ovary. These seed “fruits” are considered more primitive than hardwoods. Softwood trees are usually evergreen, bear cones, and have needles or scale like leaves. They include conifer species e.g. pine, spruces, firs, and cedars. Wood hardness varies among the conifer species.

The term hardwood is used to describe wood from trees that belong to angiosperm family. Angiosperms are plants with ovules enclosed for protection in an ovary. When fertilized, these ovules develop into seeds. The hardwood trees are usually broad-leaved; in temperate and boreallatitudes they are mostly deciduous, but in tropics and subtropics mostly evergreen. These leaves can be either simple (single blades) or they can be compound with leaflets attached to a leaf stem. Although variable in shape all hardwood leaves have a distinct network of fine veins. The hardwood plants include e.g. Aspen, Birch, Cherry, Maple, Oak and Teak.

Therefore a preferred ligno-cellulosic biomass may be selected from the group consisting of the grasses and woods. A preferred ligno-cellulosic biomass may be selected from the group consisting of the plants belonging to the conifers, angiosperms, Poaceae and/or Gramineae families. Another preferred lignocellulosic biomass may also be that biomass having at least 10% by weight of it dry matter as cellulose, or more preferably at least 5% by weight of its dry matter as cellulose.

The ligno-cellulosic biomass will also comprise carbohydrate(s) selected from the group of carbohydrates based upon the glucose, xylose, and mannose monomers. Being derived from ligno-cellulosic biomass, means that the ligno-cellulosic biomass of the feed stream will comprise glucans and xylans and lignin.

Glucans include the monomers, dimers, oligomers and polymers of glucan in the ligno-cellulosic biomass. Of particular interest is 1,4 beta glucan which is particular to cellulose, as opposed to 1,4 alpha glucan. The amount of 1,4 beta glucan(s) present in the water insoluble pre-treated ligno-cellulosic biomass should be at least 5% by weight of the water insoluble pre-treated ligno-cellulosic biomass on a dry basis, more preferably at least 10% by weight of the water insoluble pre-treated ligno-cellulosic biomass on a dry basis, and most preferably at least 15% by weight of the water insoluble pre-treated ligno-cellulosic biomass on a dry basis. Xylans include the monomers, dimers, oligomers and polymers of xylan in the water insoluble pre-treated ligno-cellulosic biomass composition.

While the water insoluble pre-treated ligno-cellulosic biomass can be free of starch, substantially free of starch, or have a starch content of 0. Starch, if present, can be less than 75% by weight of the dry content. There is no preferred starch range as its presence is not believed to affect the hydrolysis to glucose. Ranges for the starch amount, if present, are between 0 and 75% by weight of the dry content, 0 to 50% by weight of the dry content, 0 to 30% by weight of the dry content and 0 to 25% by weight of the dry content.

Because this invention is to hydrolysis of glucose, the specification and inventors believe that any ligno-cellulosic biomass with 1,4 beta glucans can be used as a feed stock for this improved hydrolysis process.

The pre-treatment process used on the naturally occurring ligno-cellulosic biomass can be any pre-treatment process known in the art and those to be invented in the future, or the pre-treatment can be a series of processes.

The ligno-cellulosic biomass feedstock may also be from woody plants. A woody plant is a plant that uses wood as its structural tissue. These are typically perennial plants whose stems and larger roots are reinforced with wood produced adjacent to the vascular tissues. The main stem, larger branches, and roots of these plants are usually covered by a layer of thickened bark. Woody plants are usually either trees, shrubs, or lianas. Wood is a structural cellular adaptation that allows woody plants to grow from above ground stems year after year, thus making some woody plants the largest and tallest plants.

These plants need a vascular system to move water and nutrients from the roots to the leaves (xylem) and to move sugars from the leaves to the rest of the plant (phloem). There are two kinds of xylem: primary that is formed during primary growth from procambium and secondary xylem that is formed during secondary growth from vascular cambium. What is usually called “wood” is the secondary xylem of such plants.

The two main groups in which secondary xylem can be found are:
1) conifers (Coniferae): there are some six hundred species of conifers. All species have secondary xylem, which is relatively uniform in structure throughout this group. Many conifers become tall trees: the secondary xylem of such trees is marketed as softwood.
2) angiosperms (Angiospermae): there are some quarter of a million to four hundred thousand species of angiosperms. Within this group secondary xylem has not been found in the monocots (e.g Poaceae). Many non-monocot angiosperms become trees, and the secondary xylem of these is marketed as hardwood.

The term softwood is used to describe wood from trees that belong to gymnosperms. The gymnosperms are plants with naked seeds not enclosed in an ovary. These seed “fruits” are considered more primitive than hardwoods. Softwood trees are usually evergreen, bear cones, and have needles or scalelike leaves. They include conifer species e.g. pine, spruces, firs, and cedars. Wood hardness varies among the conifer species.

The term hardwood is used to describe wood from trees that belong to angiosperm family. Angiosperms are plants with ovules enclosed for protection in an ovary. When fertilized, these ovules develop into seeds. The hardwood trees are usually broad-leaved; in temperate and boreallatitudes they are mostly deciduous, but in tropics and subtropics mostly evergreen. These leaves can be either simple (single blades) or they can be compound with leaflets attached to a leaf stem. Although variable in shape all hardwood leaves have a distinct network of fine veins. The hardwood plants include e.g. Aspen, Birch, Cherry, Maple, Oak and Teak.

As an example, the pre-treatment process may include soaking followed by steam explosion. For example, the pre-treatment process may include any process or processes other than steam explosion. The pre-treatment process may not include steam explosion. The pre-treatment process may include steam explosion. Steam explosion may be the last step of the pre-treatment process. Steam explosion into a flash receiver, cooling down the contents of the receiver and separating the free liquid may be the last step of the pre-treatment process. The pre-treatment process may include super-critical extraction.

The pre-treatment process used to pre-treat the water insoluble pre-treated ligno-cellulosic biomass is used to ensure that the structure of the ligno-cellulosic content is rendered more accessible to the catalysts, such as enzymes, and at the same time the concentrations of harmful inhibitory by-products such as acetic acid, furfural and hydroxymethyl furfural remain substantially low.

Some of the current strategies of pre-treatment are subjecting the ligno-cellulosic material to temperatures between 110-250° C. for 1-60 min e.g.:

Hot water extraction

Multistage dilute acid hydrolysis, which removes dissolved material before inhibitory substances are formed

Dilute acid hydrolysis at relatively low severity conditions

Alkaline wet oxidation

Steam explosion

Almost any pre-treatment with subsequent detoxification

If a hydrothermal pre-treatment is chosen, the following conditions are preferred:

Pre-treatment temperature: 110-250° C., preferably 120-240° C., more preferably 130-230° C., more preferably 140-220° C., more preferably 150-210° C., more preferably 160-200° C., even more preferably 170-200° C. or most preferably 180-200° C.

Pre-treatment time: 1-60 min, preferably 2-55 min, more preferably 3-50 min, more preferably 4-45 min, more preferably 5-40 min, more preferably 5-35 mM, more preferably 5-30 min, more preferably 5-25 min, more preferably 5-20 min and most preferably 5-15 min.

Dry matter content after pre-treatment is preferably at least 20% (w/w). Other preferable higher limits are contemplated as the amount of biomass to water in the water insoluble pre-treated ligno-cellulosic feedstock be in the ratio ranges of 1:4 to 9:1; 1:3.9 to 9:1, 1:3.5 to 9:1, 1:3.25 to 9:1, 1:3 to 9:1, 1:2.9 to 9:1, 1:2 to 9:1, 1:1.5 to 9:1, 1:1 to 9:1, and 1:0.9 to 9:1.

Polysaccharide-containing biomasses according to the present invention include any material containing polymeric sugars e.g. in the form of starch as well as refined starch, cellulose and hemicellulose. However, as discussed earlier, the starch is not a primary component.

A preferred pre-treatment process is the two steps of soaking to extract C5's followed by steam explosion as describe below.

A preferred pretreatment of a naturally occurring ligno-cellulosic biomass includes a soaking of the naturally occurring ligno-cellulosic biomass feedstock followed by a steam explosion of at least a part of the soaked naturally occurring ligno-cellulosic biomass feedstock.

The soaking occurs in a substance such as water in either vapor form, steam, or liquid form or liquid and steam together, to produce a product. The product is a soaked biomass containing a first liquid, with the first liquid usually being water in its liquid or vapor form or some mixture.

This soaking can be done by any number of techniques that expose a substance to water, which could be steam or liquid or mixture of steam and water, or, more in general, to water at high temperature and high pressure. The temperature should be in one of the following ranges: 145 to 165° C., 120 to 210° C., 140 to 210° C., 150 to 200° C., 155 to 185° C., 160 to 180° C. Although the time could be lengthy, such as up to but less than 24 hours, or less than 16 hours, or less than 12 hours, or less than 9 hours or less than 6 hours; the time of exposure is preferably quite short, ranging from 1 minute to 6 hours, from 1 minute to 4 hours, from 1 minute to 3 hours, from 1 minute to 2.5 hours, more preferably 5 minutes to 1.5 hours, 5 minutes to 1 hour, 15 minutes to 1 hour.

If steam is used, it is preferably saturated, but could be superheated. The soaking step can be batch or continuous, with or without stirring. A low temperature soak prior to the high temperature soak can be used. The temperature of the low temperature soak is in the range of 25 to 90° C. Although the time could be lengthy, such as up to but less than 24 hours, or less than 16 hours, or less than 12 hours, or less than 9 hours or less than 6 hours; the time of exposure is preferably quite short, ranging from 1 minute to 6 hours, from 1 minute to 4 hours, from 1 minute to 3 hours, from 1 minute to 2.5 hours, more preferably 5 minutes to 1.5 hours, 5 minutes to 1 hour, 15 minutes to 1 hour.

While it is preferred to avoid acid or bases, either soaking step could also include the addition of other compounds, e.g. H₂SO4, NH₃, in order to achieve higher performance later on in the process.

The product comprising the first liquid is then passed to a separation step where the first liquid is separated from the soaked biomass. The liquid will not completely separate so that at least a portion of the liquid is separated, with preferably as much liquid as possible in an economic time frame. The liquid from this separation step is known as the first liquid stream comprising the first liquid. The first liquid will be the liquid used in the soaking, generally water and the soluble species of the feedstock. These water soluble species are glucan, xylan, galactan, arabinan, glucolygomers, xyloolygomers, galactolygomers and arabinolygomers. The solid biomass is called the first solid stream as it contains most, if not all, of the solids.

The separation of the liquid can again be done by known techniques and likely some which have yet been invented. A preferred piece of equipment is a press, as a press will generate a liquid under high pressure.

It is also known to pre-soak the ligno-cellulosic biomass before soaking to remove the C5's.

The first solid stream is then steam exploded to create a steam exploded stream, comprising solids and a second liquid. Steam explosion is a well known technique in the biomass field and any of the systems available today and in the future are believed suitable for this step. The severity of the steam explosion is known in the literature as Ro, and is a function of time and temperature and is expressed as

Ro=texp[(T−100)/14.75]

with temperature, T expressed in Celsius and time, t, expressed in common units.

The formula is also expressed as Log(Ro), namely

Log(Ro)=Ln(t)+[(T−100)/14.75].

Log(Ro) is preferably in the ranges of 2.8 to 5.3, 3 to 5.3, 3 to 5.0 and 3 to 4.3.

The steam exploded stream may be optionally washed at least with water and there may be other additives used as well. It is conceivable that another liquid may be used in the future, so water is not believed to be absolutely essential. At this point, water is the preferred liquid and if water is used, it is considered the third liquid. The liquid effluent from the optional wash is the third liquid stream. This wash step is not considered essential and is optional.

The washed exploded stream is then processed to remove at least a portion of the liquid in the washed exploded material. This separation step is also optional. The term at least a portion is removed, is to remind one that while removal of as much liquid as possible is desirable (pressing), it is unlikely that 100% removal is possible. In any event, 100% removal of the water is not desirable since water is needed for the subsequent hydrolysis reaction. The preferred process for this step is again a press, but other known techniques and those not invented yet are believed to be suitable. The products separated from this process are solids in the second solid stream and liquids in the second liquid stream.

The composition for the invented process will have a dry matter content which is the material after the removal of the water and other volatiles by drying to a level of at least less than 50 ppm moisture. The dry matter content is measured by procedures disclosed in “Preparation of Samples for Compositional Analysis”, Laboratory Analytical Procedure (LAP), Issue Date: Sep. 28, 2005, Technical Report NREL/TP-510-42620, January 2008.

In one embodiment the composition prior to vacuum will have an amount of free liquid from the pre-treatment of the water insoluble pre-treated ligno-cellulosic biomass which has not been separated from the water insoluble pre-treated ligno-cellulosic biomass after the pre-treatment of the water insoluble pre-treated ligno-cellulosic biomass. For example, in some steam explosion processes, it is known that there may be free liquid from the condensed vapors. By free liquid, it is meant a liquid which can be separated from the solids of the composition by decanting the composition. If the free liquid is removed from the water insoluble pre-treated ligno-cellulosic biomass after pre-treatment, some, if not all of the free liquid can be re-added to the composition and still be within the scope of the invention.

The composition will also further comprise at least one gas, which may be air or a gas or mixture of gases used in the pre-treatment process prior to the vacuum treatment. This gas, usually air, is entrained in the solid matrix of the composition. It is this gas which is removed by the exposure of the composition to the vacuum conditions. As noted in the experimental, the expansion of the gas is substantial and is believed to open or break the pores holding the gas. The volume of the composition at atmospheric conditions after exposure to the vacuum will be less than 95% of the volume prior to exposure, with less than 90% of the volume being more preferred, and less than 85% of the volume prior to exposure even more preferred with less than 80% of the volume prior to exposure being the most preferred. One skilled in the art can control the amount of the gas removed, with 95 to 100% of the gas removal being the most preferred amount. Thus, the final composition after vacuum exposure can be void of gas, which is more than 95% of the gas having been removed.

The composition will also comprise an amount of water insoluble carbohydrates known as the amount of water insoluble carbohydrates prior to the vacuum exposure. Because the exposure to vacuum occurs before hydrolysis, the amount of the water insoluble carbohydrates prior to exposure to vacuum is expected to be the same as the amount of water insoluble carbohydrates after exposure to the vacuum.

In another embodiment the composition will be void of free liquid, in particular free liquid generated or used during the pre-treatment process. For example, a batch steam explosion may have free liquids, while a continuous steam explosion does not usually have free liquids. In another embodiment, the composition will have an amount of free liquid, but the pre-treatment process will not include a steam explosion step. The composition of this embodiment could further comprise free liquid and an added liquid as discussed below.

The composition in another embodiment further comprises an added liquid. Usually the added liquid comprises water, or is water. The amount of the added liquid depends upon the amount needed to reduce the dry matter content to the specified percentage of the total mass. The dry matter content should be the weight percent of dry matter of the composition by weight of the total amount of the composition and should be in the range of 1 to 60. Other suitable dry matter contents of the composition is a weight percent of dry matter of the composition by weight of the total amount of the composition is in a range selected from the group consisting of 1 to 50, 1 to 40, 1 to 36, 1 to 30, 1 to 25, 1 to 20, 1 to 15, 1 to 10, and 5 to 40, all expressed in weight percent of the dry matter compared to the total composition.

It is noted that the dry matter content is not just the weight of the composition less the water composition, as during the drying test, volatiles such as furfural, hydroxymethyl furfural (HMF) and acetic acid will be removed.

It is preferable that the composition be free of ammonia, added acids and/or added bases or other process reactants which have been added or used during the pre-treatment of the ligno-cellulosic biomass as they are not necessary in a properly designed pre-treatment process and create problems for downstream processing. It is also preferred that the pre-treatment process not use ammonia, added acids and/or added bases or other process reactants which have been added or used during the pre-treatment of the ligno-cellulosic biomass.

After securing the composition, the composition is exposed to a vacuum condition which could occur in any type of equipment capable of holding a vacuum. The source of vacuum could be vacuum jet(s), vacuum pump(s), ejector(s), aspirator(s), and any other vacuum source known and those to be invented yet.

One preferred method of exposing the composition to the vacuum condition is to conduct the exposure in an extruder, often called a vacuum extruder. This piece of equipment uses a screw, often called a conveying screw and/or screw, inside a cylinder to convey the composition through the vacuum zone of the cylinder apparatus.

The vacuum condition is less than atmospheric pressure which is an absolute pressure measured in millibar (mbar) less 1013.25 millibar, and can be selected from the group consisting of 950, 900, 850, 800, 700, 600, 500, 400, 300, 250, 200, 150, 100, 50, 30, 20, 10, 5, and 0.5 mBar,

The exposure of the composition to the vacuum condition may also be conducted in a temperature range consisting of a temperature range selected from the group consisting of 15 to 55° C., 15 to 50° C., 15 to 45° C., 15 to 35° C., and 15 to 30° C.

The step of exposing the composition to the vacuum condition may further include maintaining the exposure of the composition to the vacuum condition for a minimum time selected from the group consisting of 5 minutes, 10 minutes, 20 minutes, 30 minutes, 45 minutes, and 60 minutes. If a maximum exposure time is desired, the time should not be more than 600 minutes.

Because it is not necessary to conduct the catalytic hydrolysis, in particular the enzymatic hydrolysis, under a vacuum condition, the composition is preferably substantially void, or void of catalysts capable of catalytically hydrolyzing the water insoluble pre-treated ligno-cellulosic biomass. To be substantially void, means that any catalytic activity is 5% or less than the catalytic activity used in the catalytic hydrolysis step. Enzymes are known hydrolysis catalysts and in the case of enzymes, the catalytic hydrolysis is known as enzymatic hydrolysis.

It is also preferred that the added liquid comprise C5's which were separated from the water insoluble pre-treated ligno-cellulosic biomass as part of the pre-treatment of the water insoluble pre-treated ligno-cellulosic biomass prior to steam explosion. In some pre-treatment processes it is known to soak or otherwise extract the C5's, which are the arabinan and xylancomponents and include the monomers, dimers, oligomers and polymers of arabinose and xylose. This C5 removal is often done prior to steam explosion.

As it is also known to combine the water insoluble pre-treated ligno-cellulosic biomass with a product which has been previously hydrolyzed having a similar hydrolysis composition, the process may further comprise a hydrolysis product made from the enzymatic hydrolysis of a similarly composed water insoluble pre-treated ligno-cellulosic biomass, if not the hydrolysis product of the water insoluble pre-treated ligno-cellulosic biomass.

After the exposure to the vacuum condition, the vacuum is broken which is the step of ceasing to expose the composition to the vacuum condition. This can be done by isolating the vacuum source from the composition and removing the vacuum from the composition, or in the case of the extruder, moving the composition out of the vacuum zone of the extruder cylinder and into a different zone which is not under vacuum conditions or even discharging from the extruder to a tank or other vessel.

After the exposure to the vacuum condition is broken, catalytic, in particular enzymatic hydrolysis is conducted on the composition by adding at least one enzyme capable of conducting an enzymatic hydrolysis of the water insoluble pre-treated ligno-cellulosic biomass in the composition.

It is preferred that the catalytic hydrolysis is not conducted in the same vessel that the vacuum condition is conducted in. On an industrial scale the catalytic hydrolysis vessel is a large vessel. Conducting catalytic hydrolysis under vacuum would therefore require a large vessel having many moving parts for agitating the hydrolysis broth and capable of sustaining vacuum. By performing hydrolysis under vacuum additional costs would incur.

The composition may be exposed to vacuum in separated equipment in which the composition is conveyed by a screw. A person skilled in the art will recognize that this equipment is less expensive than a large vessel capable of conducting catalytic hydrolysis under vacuum. It is also contemplated that the catalytic, and in particular enzymatic hydrolysis is not done under any vacuum condition.

### EXPERIMENTAL

### Sample Preparation

Sample preparation is common to all the examples reported, if not differently explicated.

Wheat straw was subjected to a hydrothermal treatment (soaked) at a temperature of 155° C. for a time of 65 minutes and then separated into a liquid stream and a solid stream; the solid stream was steam exploded at a temperature of 190° C. for a time of 4 minutes to obtain a steam exploded solid stream. The free liquids were not separated from the steam exploded stream.

### Vacuum Treatment

Vacuum treatment was performed according to the following procedure. The sample was inserted into a vacuum vessel and sealed. The vessel was evacuated by means of a vacuum pump. Pressure reached 30 mbar in about 10 seconds and then was maintained at that level for 10 minutes.

After vacuum treatment, the vacuum was broke by venting the vessel to atmospheric pressure.

### Enzymatic Hydrolysis

Enzymatic hydrolysis is common to all examples reported, if not differently explicated.

Pretreated ligno-cellulosic biomass stream was inserted into a bioreactor, agitated by means of an impeller and heated until reaching a temperature of 50° C. pH was corrected to 5 by means of a KOH solution.

Enzymatic hydrolysis was conducted by inserting an enzymatic cocktail by Novozymes at a determined concentration of protein per gram of global cellulose contained in the pretreated stream of ligno-cellulosic biomass. In each experiment the same cocktail was used, but in different amounts.

Different enzymes concentrations were used in the experiments as indicated.

Enzymatic hydrolysis was conducted for 48 hours. Samplings were performed immediately before enzyme insertion and after a hydrolysis time of 24 hours and 48 hours from enzyme insertion.

Glucose and xylose concentration in the hydrolyzed stream was measured by means of standard HPLC.

### Example 1

A control sample was prepared at the temperature of 25° C. by mixing the liquid stream from the first pre-treatment step and steam exploded solid stream at a ratio liquid/solid ratio of 0.8, then water was added until reaching a content of 10% of dry matter on the basis of the total composition to obtain a pretreated stream of ligno-cellulosic biomass.

An amount of 1.3 Kg of pretreated stream of lignocellulosic biomass was subjected to enzymatic hydrolysis at a concentration of 5 mg of protein per gram of global cellulose contained in the pretreated stream.

A concentration of xylose of 0.956 g/l, 8.152 g/l and 8.50 g/l were measured immediately before enzyme insertion, after 24 hours and 48 hours respectively.

A concentration of glucose of 0.113 g/l, 13.934 g/l and 17.00 g/l were measured immediately before enzyme insertion, after 24 hours and 48 hours respectively.

An amount of 1.3 Kg of the pretreated ligno-cellulosic biomass stream was subjected to vacuum treatment at the temperature of 25° C. During vacuum treatment, pretreated stream expanded until reaching approximately 130% of initial volume in about 100 seconds. Macroscopic bubbles of air were formed in the pretreated stream. Shaking by hand the vacuum vessel, bubbles were removed and the pretreated stream collapsed until reaching a volume of approximately 80% of the volume of the pretreated stream before vacuum treatment. After venting, the evacuated pretreated stream was subjected to enzymatic hydrolysis at a concentration of 5 mg of protein per gram of global cellulose contained in the pretreated stream.

A concentration of xylose of 0.321 g/l, 9.800 g/l and 10.203 g/l were measured immediately before enzyme insertion, after 24 hours and 48 hours respectively. As the xylose comes from the liquid from the first pre-treatment step, its presence does not indicate enzymatic hydrolysis.

A concentration of glucose of 0 g/l, 19.426 g/l and 22.634 g/l were measured immediately before enzyme insertion, after 24 hours and 48 hours respectively. The concentration of 0 g/l after vacuum indicates that there was no hydrolysis occurring during vacuum and that water is not a process reactant.

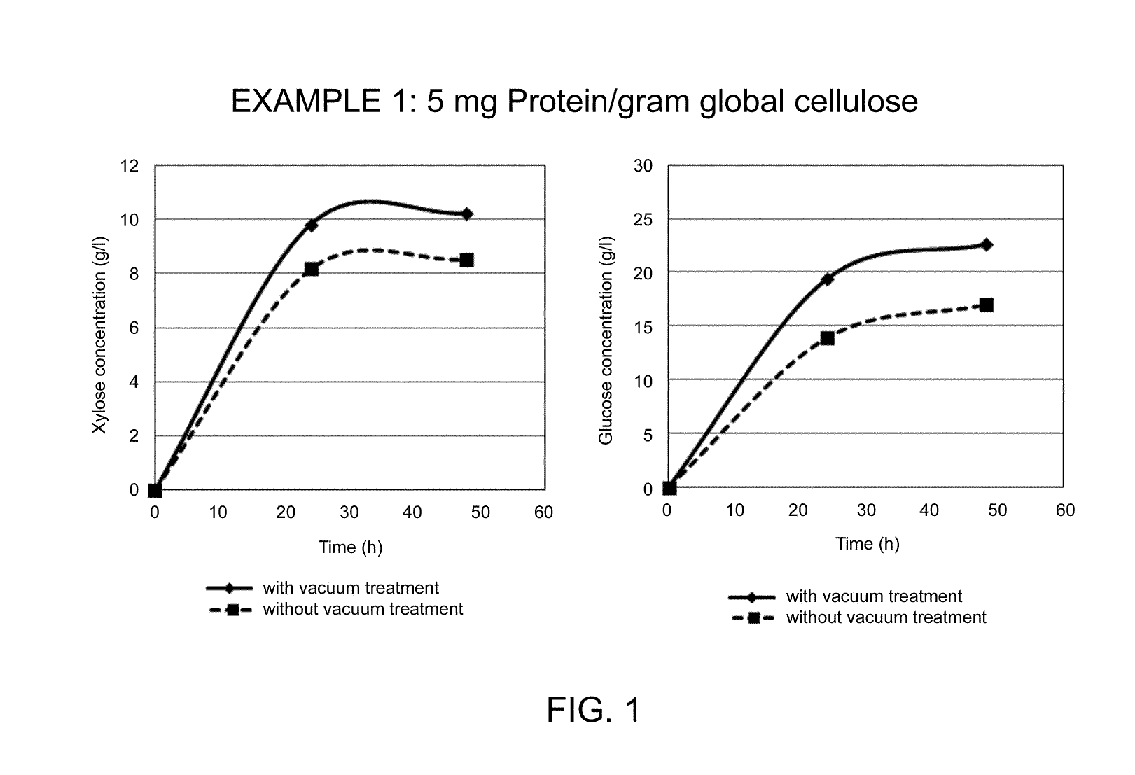

Concentrations of xylose and glucose vs. hydrolysis time for control sample and vacuum treated sample are reported in FIG. 1.

### Example 2

Using the same material as in Example 1, a control sample was prepared at the temperature of 25° C. by mixing liquid stream and steam exploded solid stream at a ratio liquid/solid ratio of 0.8, then water was added until reaching a content of 10% of dry matter to obtain a pretreated stream.

An amount of 1.3 Kg of pretreated stream was subjected to enzymatic hydrolysis at a concentration of 7.5 mg of protein per gram of global cellulose contained in the pretreated stream.

A concentration of xylose of 0.956 g/l, 9.601 g/l and 10.402 g/l were measured immediately before enzyme insertion, after 24 hours and 48 hours respectively.

A concentration of glucose of 0.113 g/l, 22.3 g/l and 28.231 g/l were measured immediately before enzyme insertion, after 24 hours and 48 hours respectively.

An amount of 1.3 Kg of pretreated stream was subjected to vacuum treatment at the temperature of 25° C. During vacuum treatment, the pretreated stream expanded until reaching approximately 130% of initial volume in about 100 seconds. Macroscopic bubbles of air were formed in the pretreated stream. Shaking by hand the vacuum vessel, bubbles were removed and the pretreated stream collapsed until reaching a volume of approximately 80% of the volume of the pretreated stream before vacuum treatment. After venting, the evacuated pretreated stream was subjected to enzymatic hydrolysis at a concentration of 7.5 mg of protein per gram of global cellulose contained in the pretreated stream.

A concentration of xylose of 0.451 g/l, 11.185 g/l and 12.052 g/l were measured immediately before enzyme insertion, after 24 hours and 48 hours respectively.

A concentration of glucose of 0 g/l, 28.201 g/l and 33.293 g/l were measured immediately before enzyme insertion, after 24 hours and 48 hours respectively.

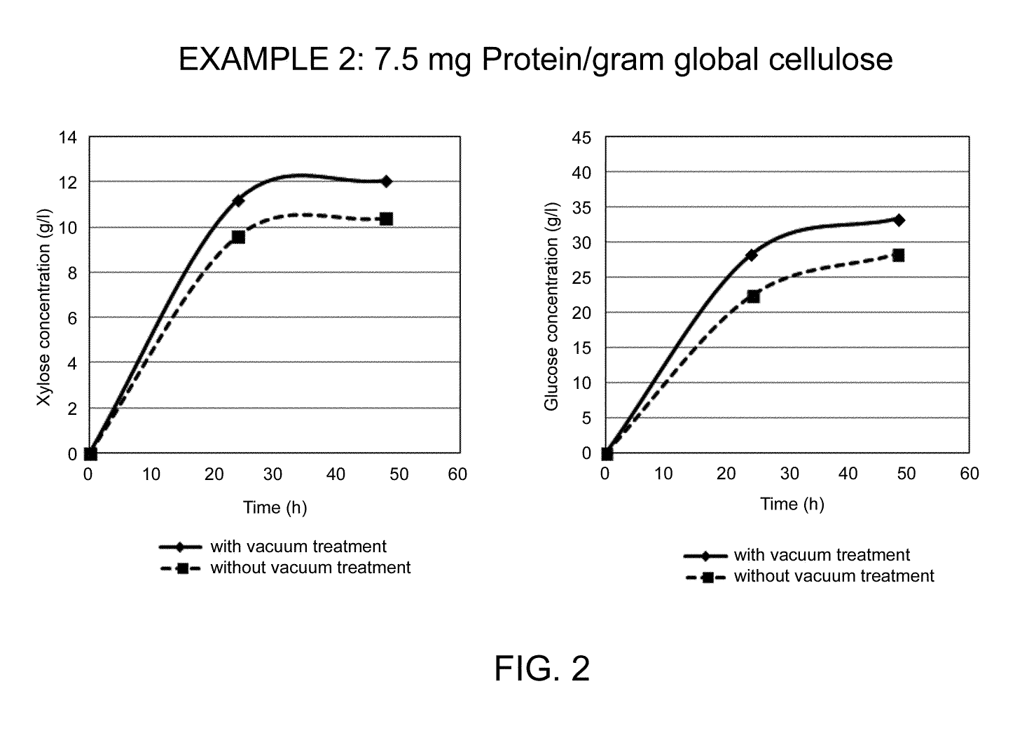

Concentrations of xylose and glucose vs. hydrolysis time for control sample and vacuum treated sample are reported in FIG. 2.

### Example 3

A control sample of the same ligno-cellulosic biomass as that used in Examples 1 and 2 was prepared at the temperature of 25° C. by mixing liquid stream and steam exploded solid stream at a liquid/solid ratio of 0.8, then water was added until reaching a content of 10% of dry matter to obtain a pretreated stream.

An amount of 1.3 Kg of pretreated material was subjected to enzymatic hydrolysis at a concentration of 10 mg of protein per gram of global cellulose contained in the pretreated stream.

A concentration of xylose of 0.956 g/l, 10.495 g/l and 11.31 g/l were measured immediately before enzyme insertion, after 24 hours and 48 hours respectively.

A concentration of glucose of 0.113 g/l, 27.325 g/l and 33.731 g/l were measured immediately before enzyme insertion, after 24 hours and 48 hours respectively.

An amount of 1.3 Kg of pretreated stream was subjected to vacuum treatment at the temperature of 25° C. During vacuum treatment, the pretreated stream expanded until reaching approximately 130% of initial volume in about 100 seconds. Macroscopic bubbles of air were formed in the pretreated stream. Shaking by hand the vacuum vessel, bubbles were removed and the pretreated stream collapsed until reaching a volume of approximately 80% of the volume of the pretreated stream before vacuum treatment. After venting, the evacuated pretreated stream was subjected to enzymatic hydrolysis at a concentration of 10 mg of protein per gram of global cellulose contained in the pretreated stream.

A concentration of xylose of 0.418 g/l, 12.698 g/l and 13.504 g/l were measured immediately before enzyme insertion, after 24 hours and 48 hours respectively.

A concentration of glucose of 0 g/l, 34.851 g/l and 39.596 g/l were measured immediately before enzyme insertion, after 24 hours and 48 hours respectively.

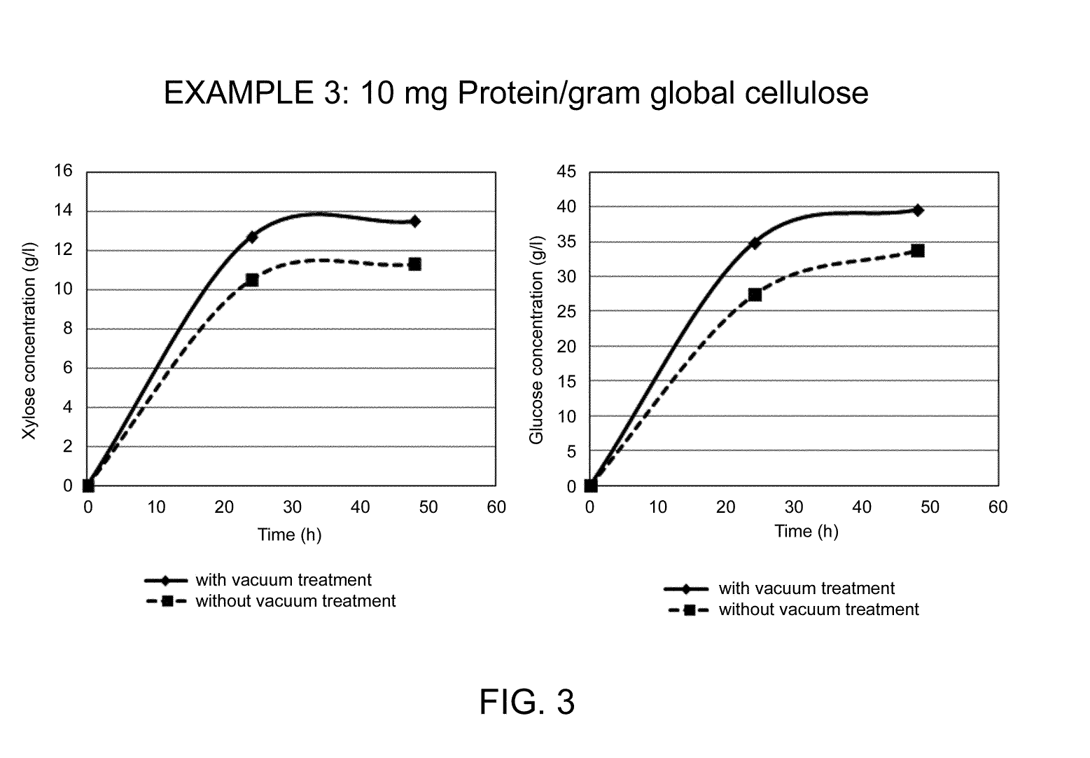

Concentrations of xylose and glucose vs. hydrolysis time for control sample and vacuum treated sample are reported in FIG. 3.

### Example 4

The control experiment corresponds to the sample of example 3, where the pretreated stream is exposed to vacuum before enzyme insertion.

An amount of 1.3 Kg of pretreated stream was added with the enzymatic cocktail by Novozymes at the concentration of 10 mg of protein per gram of global cellulose contained in the pretreated stream at the temperature of 25° C. and then subjected to vacuum treatment. During vacuum treatment, the pretreated stream expanded until reaching approximately 130% of initial volume in about 100 seconds. Macroscopic bubbles of air were formed in the pretreated stream. Shaking by hand the vacuum vessel, bubbles were removed and the pretreated stream collapsed until reaching a volume of approximately 80% of the volume of the pretreated stream before vacuum treatment. After venting, the pretreated stream with already added enzymatic cocktail was inserted into a bioreactor, agitated by means of an impeller and heated until reaching a temperature of 50° C. pH was corrected to 5 by means of a KOH solution.

Enzymatic hydrolysis was conducted for 48 hours. Samplings were performed immediately before the insertion into the bioreactor and after a hydrolysis time of 24 hours and 48 hours from enzyme insertion.

A concentration of xylose of 7.23 g/l, 12.698 g/l and 12.805 g/l was measured immediately before insertion into the bioreactor, after 24 hours and 48 hours respectively.

A concentration of glucose of 3.373 g/l, 31.498 g/l and 35.971 g/l was measured immediately before insertion into the bioreactor, after 24 hours and 48 hours respectively. Since the glucose concentration is not 0, it is indicative of enzymatic hydrolysis, but this hydrolysis has occurred after the addition of the enzymes at atmospheric pressure, indicating that the enzymatic hydrolysis does not need to be conducted under a vacuum condition as indicated in the art.

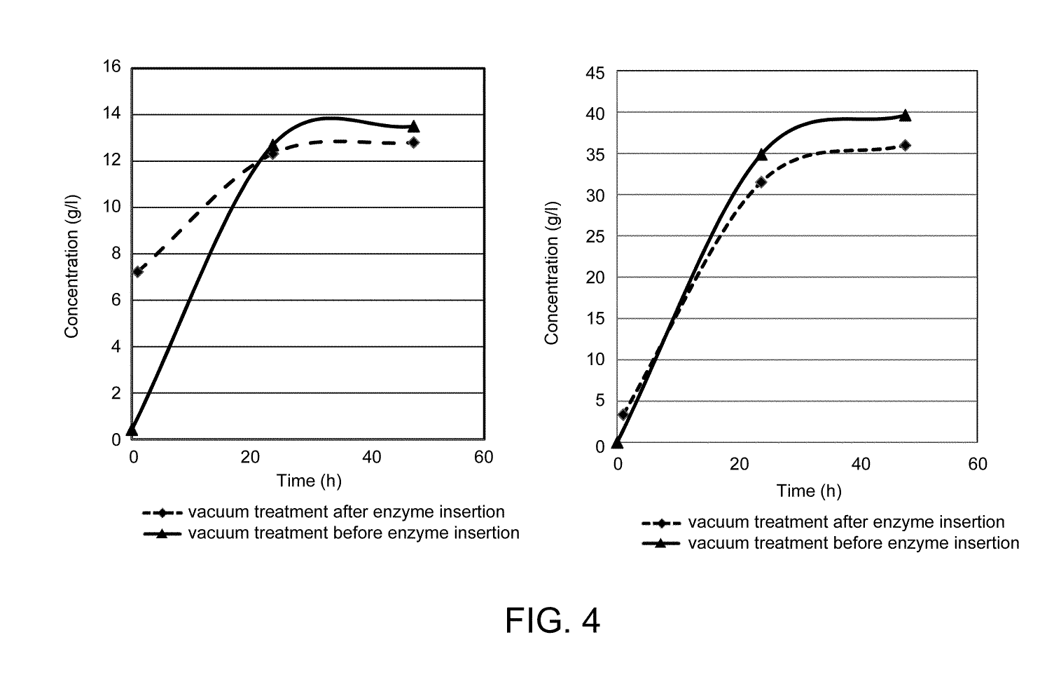

Concentrations of xylose and glucose vs. hydrolysis time for the sample exposed to vacuum before enzyme insertion and the sample exposed to vacuum after enzyme insertion (vacuum hydrolysis) are reported in FIG. 4. These results show that exposing to vacuum the pretreated stream without the enzymes is superior to exposing to vacuum the pretreated stream with the enzymes already added; in other words, surprisingly, using vacuum to penetrate the reactant does not work as well as using vacuum, removing the air and then adding the process reactant.

### Example 5

Experiment was conducted on a different source of wheat straw raw material with respect to previously reported experiments.

A control sample was prepared at the temperature of 25° C. by mixing the liquid stream from the first pre-treatment and the steam exploded solid stream at a liquid/solid ratio of 0.8, then water was added until reaching a content of 10% of dry matter to obtain a pretreated stream.

An amount of 1.3 Kg of pretreated material was subjected to enzymatic hydrolysis at a concentration of 10 mg of protein per gram of global cellulose contained in the pretreated stream.

An amount of 1.3 Kg of pretreated stream was subjected to vacuum treatment at the temperature of 25° C. During vacuum treatment, the pretreated stream expands until reaching approximately 130% of initial volume in about 100 seconds. Macroscopic bubbles of air were formed in the pretreated stream. Shaking by hand the vacuum vessel, bubbles were removed and the pretreated stream collapsed until reaching a volume of approximately 80% of the volume of the pretreated stream before vacuum treatment. After venting, the evacuated pretreated stream of ligno-cellulosic biomass was subjected to enzymatic hydrolysis at a concentration of 10 mg of protein per gram of global cellulose contained in the pretreated stream.

Enzymatic hydrolysis was conducted for a long run of 144 hours. Samplings were performed immediately before the insertion into the bioreactor and after a hydrolysis time of 6, 24, 48, 72, 96, 120 and 144 hours from enzyme insertion.

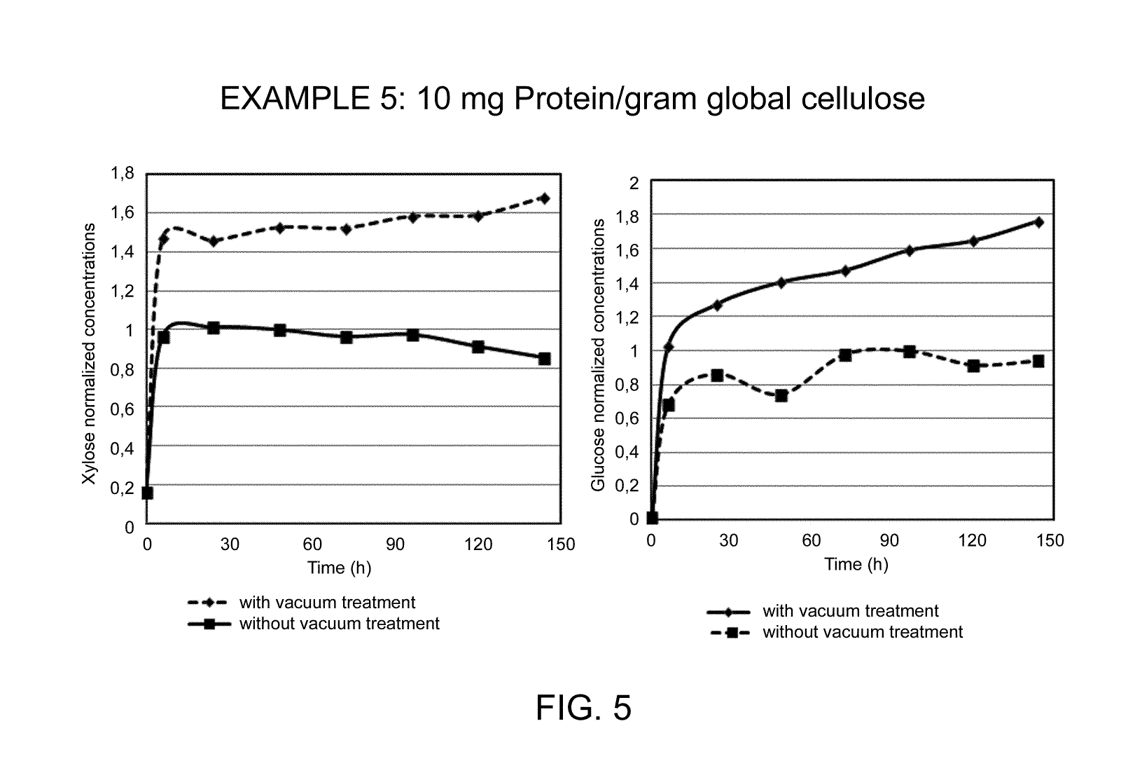

Normalized concentrations of xylose and glucose vs. hydrolysis time for control sample and vacuum treated sample are reported in FIG. 5.

This data shows the large relative amount of xylose and glucose which is converted when the material has only been exposed to vacuum in the presence of water and the liquid from the pre-treatment, and at least some of the free liquid after steam explosion has not been separated from the steam exploded stream.

We claim:

1-49. (canceled)

50. A process to increase the recovery of glucose from a pre-treated ligno-cellulosic biomass, comprising the steps of
A) Exposing a composition to a vacuum condition,
wherein the composition has a dry matter content, and
the composition comprises a water insoluble pre-treated ligno-cellulosic biomass produced from a ligno-cellulosic biomass processed in a pre-treatment process, and
an added liquid which has been added to the water insoluble pre-treated ligno-cellulosic biomass after the pre-treatment process,
wherein the weight percent of the dry matter content of the composition by weight of the total amount of the composition is in the range of 1 to 60 weight percent;
B) Ceasing to expose the composition to the vacuum condition,
C) Adding at least one catalyst to the composition wherein the catalyst is capable of hydrolyzing the water insoluble pre-treated ligno-cellulosic biomass in the composition,
D) Conducting a catalytic hydrolysis of the water insoluble pre-treated ligno-cellulosic biomass in the composition.

51. The process of claim 50, wherein the step A) of exposing the composition to a vacuum condition and the step D) of conducting the catalytic hydrolysis are not conducted in the same vessel.

52. The process of claim 50, wherein first added liquid comprises C5's which were separated from the water insoluble pre-treated ligno-cellulosic biomass as part of the pre-treatment process used to pre-treat the water insoluble pre-treated ligno-cellulosic biomass.

53. The process of claim 50, wherein the added liquid further comprises a hydrolysis product made from the catalytic hydrolysis of a similarly composed pre-treated ligno-cellulosic biomass.

54. The process of claim 50, wherein the step of exposing the composition to the vacuum condition is conducted while the composition is being conveyed with a screw.

55. The process of claim 50, wherein the process is a continuous process.

56. The process of claim 50, wherein the composition prior to exposure to the vacuum condition is void of ammonia.

57. The process of claim 50, wherein the pre-treatment process did not use ammonia to pre-treat the ligno-cellulosic biomass.

58. A process to increase the recovery of glucose from a pre-treated ligno-cellulosic biomass, comprising the steps of
A) Exposing a composition to a vacuum condition,
wherein the composition has a dry matter content, and
the composition comprises water insoluble pre-treated ligno-cellulosic biomass,
wherein the weight percent of the dry matter content of the composition by weight of the total amount of the composition is in the range of 1 to 60 weight percent, and
the composition is void of free liquid;
B) Ceasing to expose the composition to the vacuum condition;
C) Adding at least one catalyst to the composition wherein the catalyst is capable of hydrolyzing the water insoluble pre-treated ligno-cellulosic biomass in the composition;
D) Conducting a catalytic hydrolysis of the water insoluble pre-treated ligno-cellulosic biomass in the composition.

59. The process of claim 58, wherein the step of exposing the composition to the vacuum condition is conducted using a cylinder with a screw inside the cylinder.

60. The process of claim 58, wherein the process is a continuous process.

61. The process of claim 58, wherein the composition prior to exposure to the vacuum condition is void of ammonia.

62. The process of claim 58, wherein the pre-treatment process did not use ammonia to pre-treat the ligno-cellulosic biomass.

63. A process to increase the recovery of glucose from a pre-treated ligno-cellulosic biomass, comprising the steps of
A) Exposing a composition to a vacuum condition,
wherein the composition has a dry matter content, and
the composition comprises water insoluble pre-treated ligno-cellulosic biomass, and
a free liquid,
wherein the weight percent of the dry matter content of the composition by weight of the total amount of the composition is in the range of 1 to 60 weight percent;
B) Ceasing to expose the composition to the vacuum condition,
C) Adding at least one catalyst to the composition wherein the catalyst is capable of hydrolyzing the water insoluble pre-treated ligno-cellulosic biomass in the composition,
D) Conducting a catalytic hydrolysis of the water insoluble pre-treated ligno-cellulosic biomass in the composition.

64. The process of claim 63, wherein the step of exposing the composition to the vacuum condition is conducted using a cylinder with a screw inside the cylinder.

65. The process of claim 63, wherein the process is a continuous process.

66. The process of claim 63, wherein the composition prior to exposure to the vacuum condition is void of ammonia.

67. The process of claim 63, wherein the pre-treatment process did not use ammonia to pre-treat the ligno-cellulosic biomass.
